(19) United States
(12) Patent Application Publication
(10) Pub. No.: US 2025/0180530 A1
(43) Pub. Date: Jun. 5, 2025

(54) ODOR DETECTION SYSTEM AND ODOR DETECTION METHOD

(71) Applicant: Panasonic Intellectual Property Management Co., Ltd., Osaka (JP)

(72) Inventor: Chiaki SHIDO, Kyoto (JP)

(21) Appl. No.: 18/840,817

(22) PCT Filed: Feb. 22, 2023
(86) PCT No.: PCT/JP2023/006422

§ 371 Date: Aug. 22, 2024

(30) Foreign Application Priority Data
Feb. 28, 2022 (JP) 2022-030357

(51) Int. Cl. G01N 33/00; G01N 27/12; G08B 3/10

(52) U.S. Cl. CPC G01N 33/0027; G01N 27/12; G08B 3/10

(57) ABSTRACT

An odor detection system includes a sensor module, an acquirer, an identifier and an outputter. The sensor module includes: a sensitive unit having an electrical characteristic value varying in response to that the sensitive unit is exposed to an odor gas; and a housing in which the sensitive unit is housed, the housing being attached to a user. The acquirer is configured to acquire, as an output signal of the sensitive unit, data about the electrical characteristic value of the sensitive unit from the sensor module. The identifier is configured to input, to a learned model, the output signal acquired by the acquirer to identify a type of the odor gas. The outputter is configured to output notification information representing an identification result obtained by the identifier.

### TECHNICAL FIELD

The present disclosure generally relates to odor detection systems and odor detection methods. More particularly, the present disclosure relates to an odor detection system and an odor detection method, which detect odor present around a user.

### BACKGROUND ART

Patent Literature 1 discloses a gas alarm configured to alert a user wearing a helmet by inputting an alarm signal, output from a gas alarm main body attached to the helmet, to a bone conduction speaker mounted on an inner surface of the helmet. The gas alarm makes a gas sensor detect a concentration of target gas to be detected. The gas alarm also makes the bone conduction speaker output the alarm signal, when finding that the concentration of the target gas exceeds a reference value.

The human sense of smell may decline due to an effect of aging, disease, etc., or may be made insensible by prolonged exposure to the same odor. Therefore, there has been a desire to provide assistance in a function about the sense of smell with respect to various types of odor.

### CITATION LIST

### Patent Literature

Patent Literature 1: JP 2008-176457 A

### SUMMARY OF INVENTION

It is therefore an object of the present disclosure to provide an odor detection system and an odor detection method, all of which can provide assistance in a function about the sense of smell with respect to various types of odor gases.

An odor detection system according to an aspect of the present disclosure includes a sensor module, an acquirer, an identifier and an outputter. The sensor module includes: a sensitive unit having an electrical characteristic value varying in response to that the sensitive unit is exposed to an odor gas; and a housing in which the sensitive unit is housed, the housing being attached to a user. The acquirer is configured to acquire, as an output signal of the sensitive unit, data about the electrical characteristic value of the sensitive unit from the sensor module. The identifier is configured to input, to a learned model, the output signal acquired by the acquirer to identify a type of the odor gas. The learned model is generated by machine learning using, as input data, two or more output signals of the sensitive unit obtained in two or more states where the sensitive unit is exposed to two or more types of odor gases, respectively. The outputter is configured to output notification information representing an identification result obtained by the identifier.

An odor detection method according to an aspect of the present disclosure includes an acquisition step, an identification step and an output step. The acquisition step includes acquiring, as an output signal of a sensitive unit, data about an electrical characteristic value of the sensitive unit from a sensor module. The sensor module includes: the sensitive unit having the electrical characteristic value varying in response to that the sensitive unit is exposed to an odor gas; and a housing in which the sensitive unit is housed, the housing being attached to a user. The identification step includes inputting, to a learned model, the output signal acquired in the acquisition step to identify a type of the odor gas. The learned model is generated by machine learning using, as input data, two or more output signals of the sensitive unit obtained in two or more states where the sensitive unit is exposed to two or more types of odor gases, respectively. The output step includes outputting notification information representing an identification result obtained in the identification step.

### BRIEF DESCRIPTION OF DRAWINGS

FIG. 1 illustrates a schematic system configuration for an odor detection system according to an exemplary embodiment of the present disclosure;

FIG. 2 illustrates a schematic perspective view of an appearance of a sensor module included in the odor detection system;

FIG. 3 illustrates a use state of the odor detection system;

FIG. 4 schematically illustrates a sensitive unit included in the odor detection system;

FIG. 5 schematically illustrates a state of the sensitive unit before the sensitive unit absorbs odor molecules and a state of the sensitive unit after the sensitive unit has absorbed the odor molecules;

FIG. 6 is a waveform chart showing a current flowing through a heater included in the odor detection system, a temperature of the sensitive unit, an output of a negative characteristic sensitive element, and an output of a positive characteristic sensitive element;

FIG. 7 illustrates a process in which the sensitive unit included in the odor detection system provides output data;

FIG. 8 illustrates a detection area where the odor detection system detects an odor gas;

FIG. 9 is a flowchart showing operation of the odor detection system;

FIG. 10 illustrates a schematic perspective view of an appearance of a sensor module included in an odor detection system according to a first variation;

FIG. 11 illustrates a use state of an odor detection system according to a second variation;

FIG. 12 illustrates one example of a notification module included in an odor detection system according to a third variation;

FIG. 13 illustrates a use state of the odor detection system according to the third variation; and

FIG. 14 illustrates a use state of an odor detection system according to a fourth variation.

### DESCRIPTION OF EMBODIMENTS

An embodiment of the present disclosure will now be described with reference to the accompanying drawings as needed. Note that the embodiment to be described below is only an exemplary one of various embodiments of the present disclosure and should not be construed as limiting. The relative positions of respective constituent elements in upward, downward, rightward, and leftward directions are supposed to be defined as shown on the drawings unless otherwise stated. The drawings to be referred to in the following description of embodiments are all schematic representations. Thus, the ratio of the dimensions (including thicknesses) of respective constituent elements illustrated on the drawings does not always reflect their actual dimensional ratio. Also, the dimensional ratio of the respective elements is not limited to the ratio shown on the drawings.

As for each of the materials exemplified in the present description, the material may be used by itself or may also be used in combination with at least one more of the materials, whichever is appropriate. As for the content of each component included in a composition, if there are multiple substances corresponding to that component in the composition, the content of the component means the total content of those substances included in the composition.

### EMBODIMENT

### (1) Overview

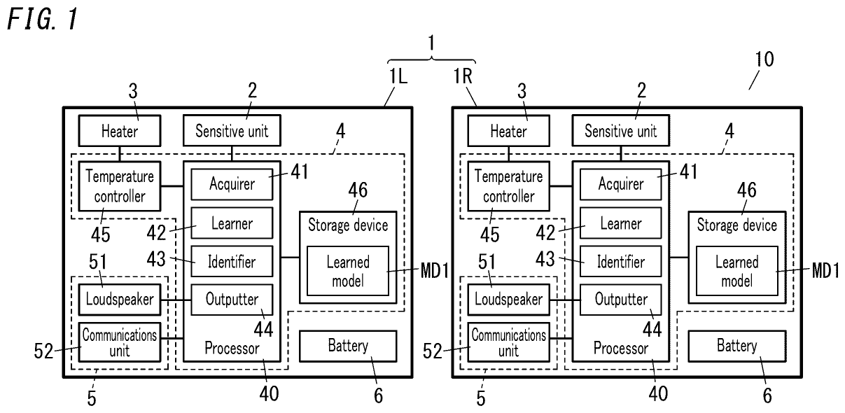

FIG. 1 illustrates a schematic system configuration for an odor detection system 10 according to an exemplary embodiment.

The odor detection system 10 includes, for example, a sensor module 1 to be attached to a user and is used to detect a type of an odor gas (i.e., a type of odor) present around the user to which the sensor module 1 is attached. The target odor to be detected includes odor molecules. Examples of the odor molecules may include volatile organic compounds (VOCs) (such as benzaldehyde, nonanal, or pyrrole), and ammonia. The target odor may include human body odor. The target odor may also include odor present in the user's living environment, e.g., odor of food, drinking water or dishes, etc., (or odor of spoilage of food, drinking water or dishes). The target odor may also include odor of flowers, soaps, shampoos, hair dressings, perfumes, or fragrances. The target odor may also include odor of a chemical generated by a volatile chemical, odor generated after something burns or when something is burning, or odor of a harmful gas, etc. The “type of the odor gas” in this embodiment may be a type of odor that can be smelled by the human sense of smell, or a type of a gas component that constitutes the odor gas.

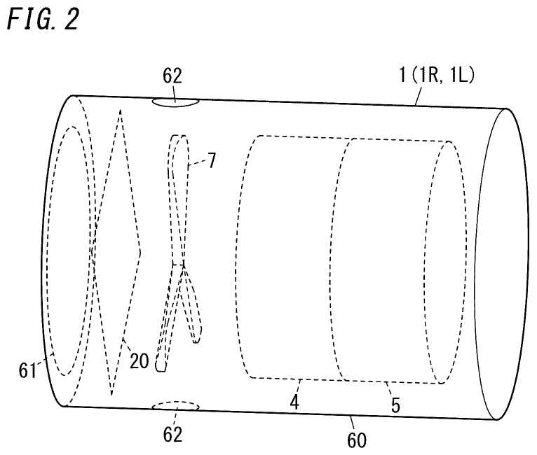

The odor detection system 10 includes a sensor module 1, an acquirer 41, an identifier 43, and an outputter 44. The sensor module 1 includes: a sensitive unit 2 having an electrical characteristic value varying in response to that the sensitive unit 2 is exposed to an odor gas; and a housing 60 (refer to FIG. 2) in which the sensitive unit 2 is housed. The housing 60 is attached to a user. The acquirer 41 is configured to acquire, as an output signal of the sensitive unit 2, data about the electrical characteristic value of the sensitive unit 2 from the sensor module 1. The identifier 43 is configured to input, to a learned model MD1, the output signal acquired by the acquirer 41 to identify a type of the odor gas. The learned model MD1 is generated by machine learning using, as input data, two or more output signals of the sensitive unit 2 obtained in two or more states where the sensitive unit 2 is exposed to two or more types of odor gases, respectively. The outputter 44 is configured to output notification information representing an identification result obtained by the identifier 43.

In this embodiment, “identifying a type of an odor gas” means determining (estimating) which of a plurality of types of odor identifiable by the human sense of smell the odor of the odor gas to be detected corresponds to. If the odor of the odor gas to be detected is a compound odor including two or more types of odor, the identifier 43 may identify all of the two or more types of odor included in the compound odor, or some of the two or more types of odor (e.g., the strongest odor in the compound odor). Alternatively, the identifier 43 may perform not only identifying the type of the odor gas but also specifying the intensity of the odor. Still alternatively, the identifier 43 may identify the type of the odor gas (e.g., fragrant odor, pungent odor, etc.) by specifying one or more types of odor molecules present in the odor gas.

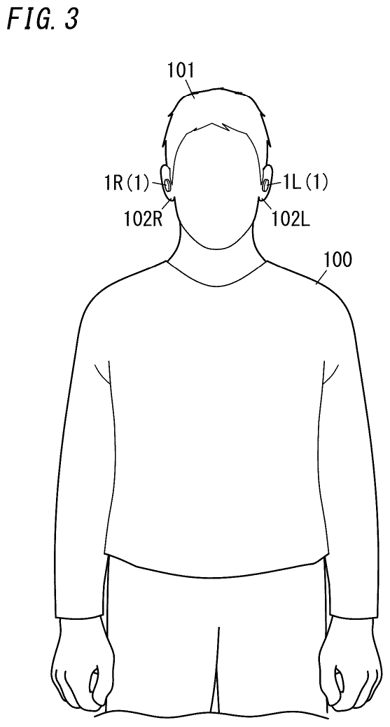

In this embodiment, in response to that the user 100 (refer to FIG. 3) acts with the sensor module 1 attached, the sensitive unit 2 housed in the housing 60 is exposed to air (odor gas) around the user 100, thereby the electrical characteristic value of the sensitive unit 2 varying. At this time, the identifier 43 inputs the output signal of the sensitive unit 2 acquired by the acquirer 41 into the learned model MD1 to identify the type of the odor gas, and then the outputter 44 outputs the identification result obtained by the identifier 43. In this manner, the odor detection system 10 identifies the type of the odor gas present around the user 100 and notifies the user 100 of the identification result. Therefore, the user 100 can know the type of the odor gas present around himself/herself even if his/her sense of smell declines or becomes insensitive. Accordingly, in this embodiment, the odor detection system can be provided, which is capable of providing assistance in a function about the sense of smell with respect to various types of odor gases. Furthermore, the housing 60 of the sensor module 1 is designed to be attached to the user 100, so that the odor detection system can detect the odor gas present around the user 100 even while the user 100 moves.

### (2) Details

### (2.1) Configuration

The odor detection system 10 according to this embodiment includes the sensor module 1 described above and a control module 4 having functions of the acquirer 41, the identifier 43, and the outputter 44, as shown in FIG. 1. The odor detection system 10 further includes a heater 3, a notification module 5, and a battery 6. The sensitive unit 2, the heater 3, the control module 4, and the notification module 5 operate using a power supply from the battery 6. In this embodiment, the control module 4, the heater 3, the notification module 5, and the battery 6 are housed together with the sensitive unit 2 in the housing 60 (refer to FIG. 2). That is to say, the acquirer 41, the identifier 43, and an outputter 44 are further housed in the housing 60 of the sensor module 1. In FIG. 2, the heater 3 and the battery 6 are not shown.

In this embodiment, the odor detection system 10 also includes two or more sensor modules 1 to be respectively attached to parts, which are different from each other, of the user 100. The two or more sensor modules 1 are preferably attached to a body of the user such that the two or more sensor modules 1 are disposed separately from each other by 90 degrees or more around a center line of the body of the user when the body is viewed from above (the center line may be e.g., a line penetrating up and down near a center of a head 101 of the user when the body is viewed from above).

In this embodiment, the sensor module 1 includes a first sensor module 1R and a second sensor module 1L. The first sensor module 1R and the second sensor module 1L are respectively attached to parts, which are different from each other, of the body of the user 100. Each of the first sensor module 1R and the second sensor module 1L includes the sensitive unit 2 and the housing 60. In this embodiment, the sensitive unit 2 of the first sensor module 1R will be sometimes referred to as a “first sensitive unit,” and the housing 60 of the first sensor module 1R will be sometimes referred to as a “first housing.” Also, the sensitive unit 2 of the second sensor module 1L will be sometimes referred to as a “second sensitive unit,” and the housing 60 of the second sensor module 1L will be sometimes referred to as a “second housing.”

More specifically, the first sensor module 1R and the second sensor module 1L are respectively attached to right and left ears (right ear 102R and left ear 102L) of the user 100 (refer to FIG. 3). The first sensor module 1R and the second sensor module 1L are attached to the body to be disposed separately from each other by almost 180 degrees around the center line of the body when the body is viewed from above. That is to say, the first sensor module 1R and the second sensor module 1L are attached at point-symmetrical positions around the center line of the body. The identifier 43 of the first sensor module 1R inputs, to the learned model MD1, the output signal of the first sensitive unit (i.e., the sensitive unit 2 of the first sensor module 1R) to identify the type of the odor gas to which the first sensitive unit is exposed. The identifier 43 of the second sensor module 1L inputs, to the learned model MD1, the output signal of the second sensitive unit (i.e., the sensitive unit 2 of the second sensor module 1L) to identify the type of the odor gas to which the second sensitive unit is exposed.

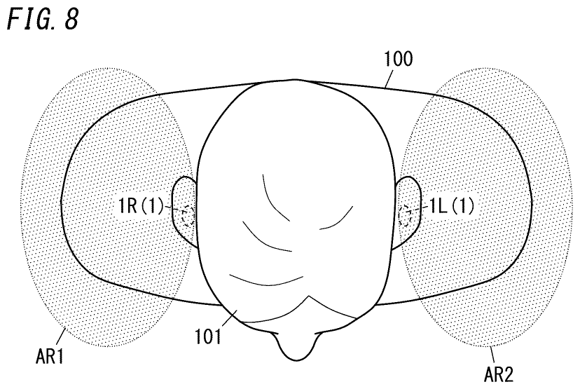

FIG. 8 shows a detection area AR1 of the first sensor module 1R and a detection area AR2 of the second sensor module 1L. In FIG. 8, the detection area AR1 and the detection area AR2 are dot-hatched areas. The odor detection system 10 including the two or more sensor modules 1 in this manner can expand the whole detection area for the odor gas. In addition, the odor detection system 10 including the two or more sensor modules 1 can specify a direction in which the odor gas is present or an area where the odor gas is present based on the detection results of the first sensor module 1R and the second sensor module 1L.

Each of the first sensor module 1R and the second sensor module 1L has the same configuration. Each of the first sensor module 1R and the second sensor module 1L is configured such that the sensitive unit 2, the heater 3, the control module 4, the notification module 5, and the battery 6 are housed in the housing 60. Since the first sensor module 1R and the second sensor module 1L are attached to be disposed separately from each other by 90 degrees or more around the center line of the body of the user 100, the odor detection system 10 can more accurately specify the direction or the area in which the odor gas is present based on the detection results of the first sensor module 1R and the second sensor module 1L.

The odor detection system 10 may include three or more sensor modules 1. The three or more sensor modules 1 are preferably attached to be in directions different from one another around the center line of the body of the user 100, and the odor detection system 10 can more finely specify the direction or the area in which the odor gas is present based on the detection results of the three or more sensor modules 1.

The configurations of the elements of the sensor module 1 (the first sensor module 1R and the second sensor module 1L) will be described below.

The control module 4 includes a processor 40, a temperature controller 45, and a storage device 46.

The processor 40 includes a control circuit configured to control the whole operation of the sensor module 1. The processor 40 may be implemented as a computer system including one or more processors (microprocessors) and one or more memories. That is to say, the functions of the processor 40 are performed by making the one or more processors execute one or more programs (applications) stored in the one or more memories. In this embodiment, the program is stored in advance in the memory of the processor 40 or the storage device 46. Alternatively, the program may also be downloaded via a telecommunications line such as the Internet or distributed after having been stored in a non-transitory storage medium such as a memory card. The processor 40 has not only the functions of the acquirer 41, the identifier 43, and the outputter 44 described above but also a function of a learner 42. In FIG. 1, none of the acquirer 41, learner 42, identifier 43, and outputter 44 has a substantive configuration but each of these units 41-44 just represents a function performed by the processor 40.

The notification module 5 includes a loudspeaker 51 for audibly outputting the notification information and a communications unit 52 for performing wireless communication with an external device (e.g., another sensor module 1 included in the odor detection system 10, or any other element). The communications unit 52 includes a wireless communications module that performs wireless communication over a short distance. The wireless communications module may use a communication system that conforms to standards such as Wi-Fi (registered trademark), Bluetooth (registered trademark), ZigBee (registered trademark), or low-power radio (specified low-power radio) that does not require a license.

As shown in FIG. 2, the housing 60 of the sensor module 1 has a hollow cylindrical shape and is formed, for example, of a synthetic resin having electrical insulation. The housing 60 is attached to the body of the user 100 by an end part of the housing 60 being inserted into an ear hole of the user 100. The housing 60 is preferably provided with a synthetic resin cover having elasticity, mounted to an outer circumference of the housing 60. The housing 60 can be stably attached to the ear hole of the user by the cover being deformed to fit the shape of the ear hole.

The housing 60 includes an intake port 61 that is provided in an end surface of the housing 60, which is exposed to the outside of the ear hole while the end part of the housing 60 is attached into the ear hole. Also, the housing 60 further includes one or more outlet ports 62 (for example, two outlet ports in FIG. 2) that are provided in a peripheral surface of the housing 60, which is exposed to the outside of the ear hole while the end part of the housing 60 is attached into the ear hole. It is preferable that a mesh-like filter or any other element be attached to the intake port 61 to prevent dust or the like from entering the intake port 61. The sensitive unit 2 is disposed in the housing 60 so as to face a flow passage through which air flows between the intake port 61 and the outlet ports 62.

The housing 60 is provided with an air blower 7 housed in the housing 60. The air blower 7 is implemented as a blower fan whose rotation is controlled by the control module 4. That is to say, the housing 60 includes the intake port 61 and the outlet port 62, and the housing 60 is provided with the air blower 7. The air blower 7 is disposed in the housing 60 to introduce an air outside of the housing 60 into the housing 60 through the intake port 61 and exhaust the air to an outside of the housing 60 through the outlet port 62. The air blower 7 preferably has a suction force equivalent to, for example, a human nose (e.g., a suction force of about 0.15 L/sec), allowing quickly detection of the odor gas present around the user 100. The air blower 7 is not limited to the blower fan but may be implemented as an air pump or any other device.

For example, the air blower 7 generates an air flow inside the housing 60 by rotating a propeller of the air blower 7. In response to the air flow generated by the air blower 7, the air outside of the housing 60 is introduced into the housing 60 through the intake port 61, and the air thus passing through the interior of the housing 60 is discharged through the outlet ports 62 to the outside of the housing 60. This allows the sensitive unit 2 housed in the housing 60 to be exposed to the air outside the housing 60 (i.e., the outside air can be brought into contact with the sensitive unit 2). Thus, the odor detection system 10 can detect the air outside the housing 60, i.e., the odor gas present around the user 100. In addition, compared to the case where the air blower 7 is not provided, the odor detection system 10 can reduce a time required for detecting the odor present around the user.

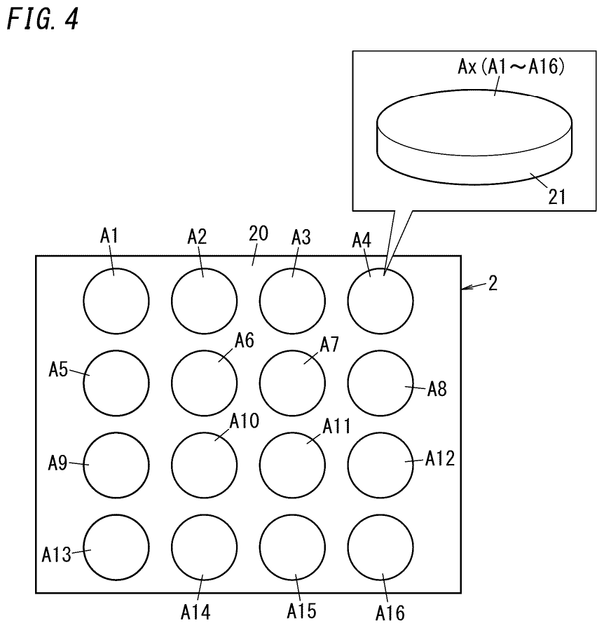

As shown in FIG. 4, the sensitive unit 2 includes a plurality of sensitive elements Ax having mutually different sensitivities. In this embodiment, the sensitive unit 2 includes sixteen sensitive elements Ax, which will be hereinafter sometimes referred to as sensitive elements A1-A16. The sixteen sensitive elements A1-A16 are arranged in four rows and four columns on one of main surfaces of a board 20 having a flat plate shape. On the other of the main surfaces of the board 20, the heater 3 is provided to heat the sensitive unit 2 disposed on the board 20. In this embodiment, the board 20 (refer to FIG. 2) (on which the sensitive unit 2 and the heater 3 are provided) is disposed in the housing 60 so as to face the flow passage through which the air passes between the intake port 61 and the outlet ports 62.

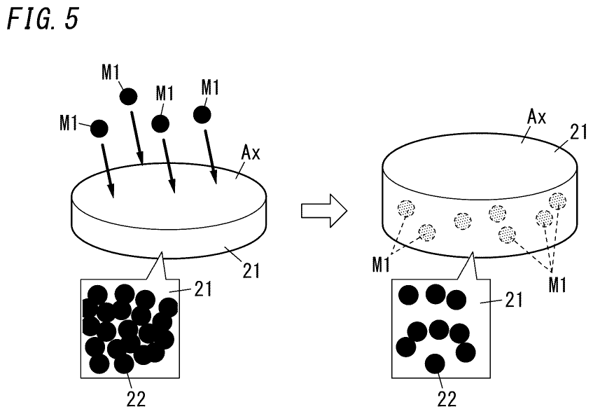

As shown in FIGS. 4 and 5, each of the plurality of the sensitive elements Ax of the sensitive unit 2 includes: an organic composition 21 formed into a disk shape out of an organic material having sensitivity to odor molecules to be included in an odor gas; and conductive particles 22 dispersed in the organic composition 21. Each of the plurality of the sensitive elements Ax is formed in a film shape. When the sensitive element Ax is exposed to the odor gas including odor molecules, the organic composition 21 absorbs the odor molecules and expands. In FIG. 5, the portion on the left-hand side illustrates a state of the sensitive element Ax that has not absorbed odor molecules M1 yet and the portion on the right-hand side illustrates a state of the sensitive element Ax that has absorbed the odor molecules M1. When the sensitive element Ax absorbs the odor molecules M1, the organic composition 21 expands. Thus, after the sensitive element Ax has absorbed the odor molecules M1, an interval between the conductive particles 22 widens, and an electrical resistance as an electrical characteristic value of the sensitive element Ax increases, compared to the interval and the electrical resistance before the sensitive element Ax absorbs the odor molecules M1.

Note that the sensitive element Ax has temperature dependence that causes the electrical characteristic value (electrical resistance) thereof to vary according to the temperature. In this case, there are two types of sensitive elements Ax, namely, a sensitive element Ax, of which the electrical resistance increases as the temperature rises and which has a positive resistance coefficient; and a sensitive element Ax, of which the electrical resistance decreases as the temperature rises and which has a negative resistance coefficient.

In this embodiment, the sensitive unit 2 includes negative characteristic sensitive elements having a negative resistance coefficient in a temperature range equal to or higher than −20° C. and equal to or lower than 50° C. The sensitive elements A1-A11 correspond to negative characteristic sensitive elements. The sensitive unit 2 also includes positive characteristic sensitive elements having a positive resistance coefficient in a temperature range equal to or higher than −20° C. and equal to or lower than 50° C. The sensitive elements A12-A16 correspond to positive characteristic sensitive elements.

The following Table 1 shows the respective compositions of the sixteen sensitive elements A1-A16. In Table 1, the percentage of the side chain shown in the “side chain feature” column indicates the proportion to the entire side chain. The compositions of the sixteen sensitive elements A1-A16 shown in Table 1 are one examples. At least some of the sixteen sensitive elements A1-A16 may be sensitive with respect to each of two or more types of odor gases to be detected. The compositions of the sixteen sensitive elements A1-A16 may be modified as appropriate.

TABLE 1

| Sensitive element | Main chain | Side chain feature |
|---|---|---|
| A1 | Siloxane | Methyl |
| A2 | Siloxane | Methyl, 10% phenyl |
| A3 | Siloxane | Methyl, cyano propyl (≤20%) |
| A4 | Siloxane | Methyl, 20% phenyl |
| A5 | Siloxane | Methyl, 35% phenyl |
| A6 | Siloxane | Methyl, 50% phenyl |
| A7 | Siloxane | Methyl, 7% phenyl, |
| | | 7% cyano propyl |
| A8 | Siloxane | Methyl, 65% phenyl |
| A9 | Siloxane | Methyl, 75% phenyl |
| A10 | Siloxane | Methyl, 50% fluoropropyl |
| A11 | Siloxane | Methyl, 25% cyano propyl, |
| | | 25% phenyl |
| A12 | Siloxane | Methoxy polyethylene glycol |
| | | propyl, phenyl |
| A13 | PEG modified | Nitro |
| | with terephthalic | |
| | acid | |
| A14 | Siloxane | 90% cyano propyl, 10% phenyl |
| A15 | Siloxane | Cyano propyl |
| A16 | Siloxane | Dicyano allyl |

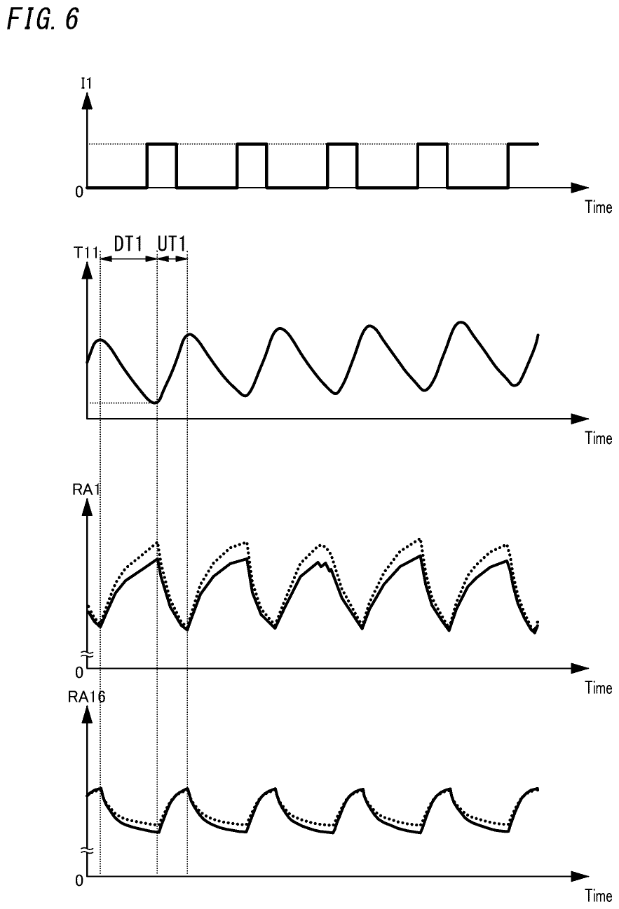

The temperature controller 45 controls the temperature of the sensitive unit 2 by causing pulses of a current I1 (refer to FIG. 6) to flow through the heater 3 in response to a control signal received from the processor 40. Causing such pulses of a current I1 to flow through the heater 3 allows the temperature of the sensitive unit 2 disposed on the board 20 to be controlled in a temperature variation pattern in which a temperature rising period UT1 in which the temperature of the sensitive unit 2 rises and a temperature falling period DT1 in which the temperature of the sensitive unit 2 falls alternate with each other repeatedly. The temperature rising period UT1 needs to be long enough to desorb the odor molecules and may be a few ten seconds, for example, but may be changed as appropriate. The temperature falling period DT1 needs to be long enough to stabilize the variation in the electrical characteristic value (e.g., a resistance value) due to adsorption of the odor molecules and may be a few ten seconds, for example, but may be changed as appropriate.

The sensitive element A1 is a negative characteristic sensitive element, of which the resistance value decreases as the temperature rises. Thus, if the temperature of the sensitive element A1 is caused to vary in the temperature variation pattern, then a resistance value RA1 of the sensitive element A1 varies in a pattern in which the resistance value RA1 decreases as the temperature rises in the temperature rising period UT1 and increases as the temperature falls in the temperature falling period DT1. In this case, if the sensitive element A1 is exposed to a gas including the odor molecules, the resistance value RA1 decreases due to desorption of the odor molecules in the temperature rising period UT1 and increases due to absorption of the odor molecules in the temperature falling period DT1. Consequently, the resistance value RA1 of the sensitive element A1 varies in a variation pattern in which variation components corresponding to the quantity of the odor molecules adsorbed into the sensitive element A1 are superposed on the variation components caused by the temperature variation. In FIG. 6, the resistance value RA1 in a situation where the sensitive element A1 is exposed to a standard gas (such as nitrogen gas) including no odor molecules is indicated by the solid curve and the resistance value RA1 in a situation where the sensitive element A1 is exposed to an odor gas including odor molecules is indicated by the dotted curve.

The sensitive element A16 is a positive characteristic sensitive element, of which the resistance value increases as the temperature rises. Thus, if the temperature of the sensitive element A16 is caused to vary in the temperature variation pattern, then a resistance value RA16 of the sensitive element A16 varies in a pattern in which the resistance value RA16 increases as the temperature rises in the temperature rising period UT1 and decreases as the temperature falls in the temperature falling period DT1. In this case, if the sensitive element A16 is exposed to an odor gas including the odor molecules, the resistance value RA16 decreases due to desorption of the odor molecules in the temperature rising period UT1 and increases due to absorption of the odor molecules in the temperature falling period DT1. Consequently, the resistance value RA16 of the sensitive element A16 varies in a variation pattern in which variation components corresponding to the quantity of the odor molecules adsorbed into the sensitive element A1 are superposed on the variation components caused by the temperature variation. In FIG. 6, the resistance value RA16 in a situation where the sensitive element A16 is exposed to a standard gas (such as nitrogen gas) is indicated by the solid curve and the resistance value RA16 in a situation where the sensitive element A16 is exposed to an odor gas including odor molecules is indicated by the dotted curve.

In this embodiment, the temperature controller 45 controls the heater 3 for heating the sensitive unit 2 in accordance with the control signal received from the processor 40 to change the temperature of the sensitive unit 2 from a first temperature that is the ambient temperature to a second temperature higher than the ambient temperature, or vice versa. The processor 40 monitors a sensing result of a temperature sensor disposed inside the housing 60 for detecting the temperature of the sensitive unit 2, for example, and controls the heater 3 based on the sensing result of the temperature sensor. Specifically, in response to the control signal received from the processor 40, the temperature controller 45 controls the heater 3 such that the temperature of the sensitive unit 2 varies in a temperature variation pattern in which a temperature rising period in which the temperature of the sensitive unit 2 increases to the second temperature and a temperature falling period in which the temperature of the sensitive unit 2 decreases to the first temperature alternate with each other repeatedly.

Note that the second temperature is set at a temperature higher by, for example, about 7° C. to about 35° C. than the first temperature. The difference between the first temperature and the second temperature is preferably wide enough to cause the odor molecules to be adsorbed or desorbed and preferably narrow enough to minimize the variation in the resistance value due to the temperature variation. In this example, the difference between the first temperature and the second temperature needs to at least equal to or greater than 7° C. and at most equal to or less than 35° C. Also, the difference between the first temperature and the second temperature is preferably equal to or greater than 20° C. and equal to or less than 35° C. and more preferably equal to or greater than 20° C. and equal to or less than 25° C.

For example, if the first temperature is 25° C., then the temperature controller 45 controls the temperature of the sensitive unit 2 between 25° C. as the first temperature and 50° C. as the second temperature. On the other hand, if the first temperature is 0° C., then the temperature controller 45 controls the temperature of the sensitive unit 2 between 0° C. as the first temperature and 25° C. as the second temperature. Note that the temperature controller 45 does not have to change the temperature of the sensitive unit 2 in such a temperature variation pattern. Rather, in a variation pattern of the output signal of the sensitive unit 2, the temperature controller 45 may change the temperature of the sensitive unit 2 in such a temperature variation pattern that causes the variation components caused due to absorption of the odor molecules to change significantly.

In this case, in a situation where the sensitive unit 2 is exposed to an odor gas including odor molecules M1, as the temperature of the sensitive unit 2 rises in the temperature rising period, the odor molecules M1 absorbed into the organic composition 21 desorb, thus causing a decrease in electrical resistance as an electrical characteristic value of the sensitive unit 2. Thereafter, as the temperature of the organic composition 21 falls in the temperature falling period, the organic composition 21 adsorbs the odor molecules M1, thus causing an increase in resistance value as an electrical characteristic value of the sensitive unit 2.

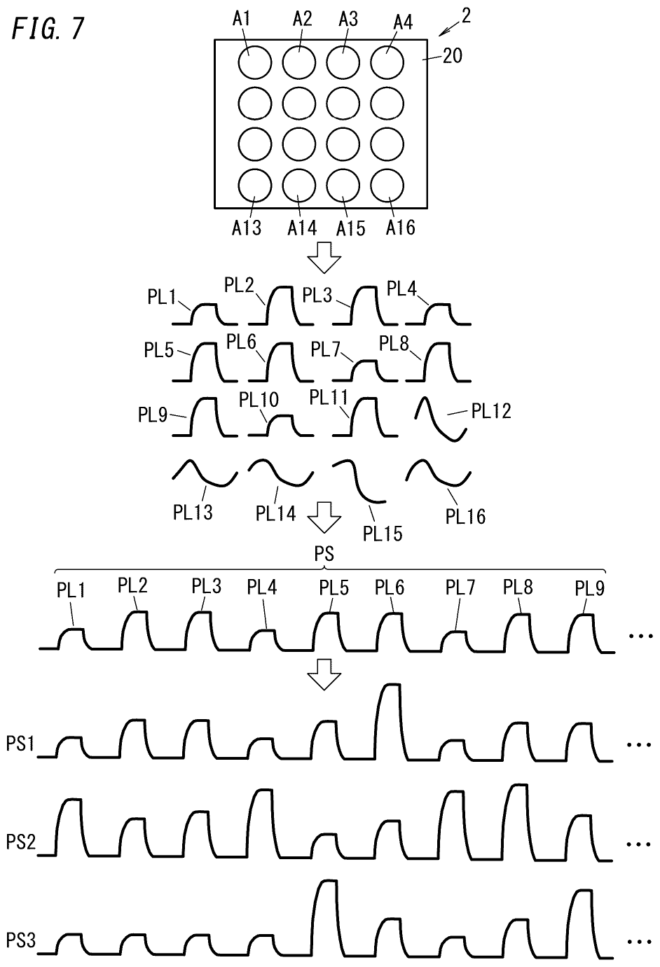

The acquirer 41 acquires, while the temperature controller 45 is supplying pulses of a current to the heater 3, data about the temperature variation pattern of the sensitive unit 2 detected by the temperature sensor and pulse outputs PL1-PL16 (refer to FIG. 7) of the sensitive elements A1-A16 for one cycle including one temperature rising period and one temperature falling period. Note that a constant DC voltage is applied to each of the sensitive elements A1-A16 and the acquirer 41 acquires the variation in the electrical resistance value of each of the sensitive elements A1-A16 as a variation in a current flowing through each of the sensitive elements A1-A16. Thus, the pulse outputs PL1-PL16 correspond to current signals, of which the magnitudes vary according to the electrical resistances of the sensitive elements A1-A16, respectively.

When acquiring the data about the temperature variation pattern of the sensitive unit 2 and pulse outputs PL1-PL16 of the sensitive elements A1-A16 for one cycle, the acquirer 41 acquires, as an output signal PS (i.e., a variation pattern of the electrical characteristic value) of the sensitive unit 2, a pulse train in which the pulse outputs PL1-PL16 are connected to each other in a predetermined order. Output signals PS1-PS3 shown in FIG. 7 are respectively examples of output signals PS in three states where the sensitive unit 2 is exposed to three types of odor gases having mutually different types of odor molecules. The sixteen sensitive elements A1-A16 exhibit mutually different sensitivities to the molecules to be detected. Thus, the output signals PS1-PS3 are pulse trains having mutually different variation patterns of the electrical characteristic value depending on the types of odor gases.

The learner 42 generates a learned model MD1. That is to say, the learner 42 is in charge of the learning phase. In the learner 42, data about the temperature variation patterns acquired by the acquirer 41 and the output signals PS of the sensitive elements A1-A16 are accumulated as learning data for use to generate the learned model MD1. The learner 42 generates the learned model MD1 based on the learning data thus collected. That is to say, the learner 42 uses the machine learning data acquired by the odor detection system 10 to make an artificial intelligence program (algorithm) learn the relation between the variation patterns (output signals PS) of the electrical characteristic values of the sensitive elements A1-A16 and types of odor gases. The artificial intelligence program is a machine learning model and may be, for example, a neural network as a type of a hierarchical model. The learner 42 makes the neural network make machine learning (such as deep learning) using the learning data, thereby generating the learned model MD1 and storing the learned model MD1 in the storage device 46. Optionally, the learner 42 may attempt to improve the performance of the learned model MD1 by making relearning using the learning data newly collected by the acquirer 41 after having generated the learned model MD1.

The storage device 46 includes one or more storage devices. Examples of the storage device include a RAM, a ROM, and an EEPROM. The storage device 46 stores, for example, the learned model MD1 for use to identify a type of an odor gas. The learned model MD1 may be generated by a learning system (i.e., the learner 42) of the odor detection system 10 making the machine learning. Alternatively, the learned model MD1 may also be generated by a learning system other than the odor detection system 10.

The identifier 43 is in charge of so-called “inference phase.” The identifier 43 uses the learned model MD1 stored in the storage device 46 to identify, based on the output signal PS acquired by the acquirer 41, a type of an odor gas. Specifically, the identifier 43 inputs, to the learned model MD1, the output signal PS of the sensitive unit 2 in a state where the temperature controller 45 controls the heater 3 such that the temperature of the sensitive unit 2 exposed to the odor gas varies in a predetermined temperature variation pattern, thereby identifying the type of the odor gas. In this embodiment, the sensitive unit 2 includes the plurality of sensitive elements Ax (namely, the sensitive elements A1-A16), and the changes in the resistance values thereof with respect to the odor molecules are different from each other. Thus, the identifier 43 identifies the type of the odor gas based on the variation patterns of the electrical characteristics of the plurality of sensitive elements Ax in a state where the temperatures of the plurality of sensitive elements Ax exposed to the odor gas are caused to vary in the temperature variation pattern. Furthermore, the identifier 43 inputs data of the temperature variation pattern of the sensitive unit 2 then acquired by the acquirer 41 and the output signal PS of the sensitive unit 2 to the learned model MD1. The learned model MD1 makes inference based on the output signal PS when the temperature of the sensitive unit 2 varies in the temperature variation pattern, thereby identifying the type of the odor gas.

In this embodiment, the identifier 43 identifies which type of odor the odor gas (to which the sensitive unit 2 is exposed) belongs to, among types of odor that can be smelled by human sense of smell. When identifying the type of the odor gas, the identifier 43 may further specify the intensity of odor as well as the type of the odor gas. Also, if the air around the user 100 is a compound odor including two or more types of odor gases, the identifier 43 may further identify some or all of the two or more types of the odor gases included in the compound odor.

It is not essential that the odor detection system 10 includes the learner 42. The identifier 43 may perform the inference phase using a learned model MD1 generated by an external computer system.

The outputter 44 outputs notification information to notify the user of an identification result obtained by the identifier 43. In this embodiment, the odor detection system 10 includes two sensor modules 1 (i.e., the first sensor module 1R and the second sensor module 1L), so the outputter 44 outputs the notification information based on identification results obtained by the two sensor modules 1.

In this embodiment, for example, one of the first sensor module 1R and the second sensor module 1L operates as a master and the other thereof operates as a slave. For example, when the first sensor module 1R operates as the master and the second sensor module 1L operates as the slave, the outputter 44 of the second sensor module 1L as the slave allows the communications unit 52 of the slave to transmit the identification result of the identifier 43 of the slave to the first sensor module 1R as the master.

When the communications unit 52 of the first sensor module 1R as the master receives the identification result of the identifier 43 from the second sensor module 1L as the slave, the processor 40 of the first sensor module 1R identifies a type of an odor gas and specifies an area where the odor gas is present based on the identification result of the identifier 43 of the first sensor module 1R and the identification result of the identifier 43 of the second sensor module 1L. If both the first sensor module 1R and the second sensor module 1L detect the same type of odor molecules, the processor 40 of the first sensor module 1R decides that the odor gas is present in both the detection area AR1 (refer to FIG. 8) of the first sensor module 1R and the detection area AR2 (refer to FIG. 8) of the second sensor module 1L. The processor 40 of the first sensor module 1R allows the outputter 44 to output, to the notification module 5, the notification information to notify the user of the type of the odor gas and the direction or the area (in this case, the entire area around the user) in which the odor gas is present, thus detected. In addition, if only either the first sensor module 1R or the second sensor module 1L detects the odor molecules, the processor 40 of the first sensor module 1R allows the outputter 44 to output, to the notification module 5, the notification information to notify the user of the type of the odor gas and the direction or the area (in this case, the detection area AR1 or the detection area AR2) in which the odor gas is present, thus detected.

In this case, a loudspeaker 51 (a first voice output unit) of the notification module 5 included in the first sensor module 1R outputs the notification information by voice and furthermore the communications unit 52 transmits the notification information to the second sensor module 1L as the slave. When the communications unit 52 of the second sensor module 1L as the slave receives the notification information from the first sensor module 1R, the processor 40 of the slave allows the outputter 44 of the slave to output the notification information to a loudspeaker 51 (a second voice output unit) of the notification module 5 of the slave, thereby the notification information being output by voice from the loudspeaker 51. That is to say, the outputter 44 outputs the notification information from the first and second voice output units (loudspeakers 51) disposed in the right and left ears of the user 100, respectively. This allows the user 100 to hear the notification information output by voice from the loudspeakers 51 of the first sensor module 1R and the second sensor module 1L attached to the right and left ears, and to know a type of an odor gas present around the user and a direction or an area in which the odor gas is present.

For example, if bouquet of wine is detected by both the first sensor module 1R and the second sensor module 1L, the outputters 44 thereof may output, by voice from the loudspeakers 51, notification information as e.g., “Bouquet of rich and full-bodied wine is present,” thereby notifying the user 100 of the type of the odor gas. Also, if burnt odor is detected by only the first sensor module 1R, out of the first sensor module 1R and the second sensor module 1L, the outputter 44 may output, by voice from the loudspeaker 51, notification information as e.g., “Burnt odor is coming from the right side,” thereby notifying the user 100 of the type of the odor gas and the direction or the area in which the odor is present.

This allows the user 100 to know a type of an odor gas present around the user 100 and to provide assistance in a function about his/her sense of smell, even if his/her sense of smell declines due to an effect of aging or disease, etc., or even if his/her sense of smell becomes insensitive due to prolonged exposure to the odor gas. Therefore, the user 100 can easily avoid dangers by knowing presence of odor such as odor of rotten things or odor of burning things. Also the user 100 can improve “Quality of Life” (QOL) by knowing presence of odor of food, drinking water, cooking, flowers, perfumes, or the like.

It is not essential that the first sensor module 1R and the second sensor module 1L perform, in cooperation with each other, the identification processing and the notification processing. Alternatively, each of the first sensor module 1R and the second sensor module 1L may individually perform the identification of the odor gas and output the notification information for notifying the user of the identification result.

The sensor module 1 (the first sensor module 1R and the second sensor module 1L) according to this embodiment further includes: a microphone for converting external sounds into electrical signals; and an amplifier for amplifying the sounds picked up by the microphone and outputting them from the loudspeaker 51. In other words, the sensor module 1 further has a hearing aid function. That is to say, the sensor module 1 has functions assisting both the human hearing and the human sense of smell. It is not essential that the sensor module 1 additionally includes the function assisting the human hearing (the hearing aid function), but the sensor module 1 is required to have at least the function assisting the human sense of smell. When the sensor module 1 is attached in the ear hole, the housing 60 preferably has a through hole or a mechanism for transmitting the external sounds so that the external sounds reach the eardrum.

### (2.2) Description of Operation

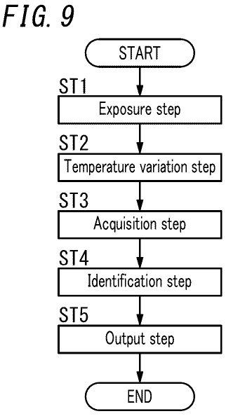

Next, it will be described with reference to the flowchart shown in FIG. 9 how the odor detection system 10 according to this embodiment operates in the inference phase. Note that the flowchart of FIG. 9 shows only an exemplary odor gas detection method according to this embodiment. Thus, the processing steps shown in FIG. 9 may be performed in a different order as appropriate, an additional processing step may be performed as appropriate, or at least one of the processing steps may be omitted as appropriate.

When the user 100 of the odor detection system 10 turns a power switch of the sensor module 1 (the first sensor module 1R and the second sensor module 1L) ON, the sensor module 1 activates and performs an operation of detecting an odor gas every predetermined period (e.g., once every few seconds to minutes).

The processor 40 of the sensor module 1 operates the air blower 7 to introduce air (odor gas) through the intake port 61 into the housing 60 such that the sensitive unit 2 is exposed to the air (odor gas) (in Exposure step ST1).

The processor 40 allows the temperature controller 45 to control the temperature of the sensitive unit 2 to be caused to vary in the temperature variation pattern in which the temperature rising period UT1 and the temperature falling period DT1 alternate with each other repeatedly (in Temperature variation step ST2).

The acquirer 41 acquires the variation pattern of the electrical characteristic value of the sensitive unit 2 in the state where the temperature of the sensitive unit 2 is caused to vary in the temperature variation pattern described above (in Acquisition step ST3).

When the variation pattern of the electrical characteristic value is acquired by the acquirer 41, the identifier 43 inputs, to the learned model MD1, the data about the variation pattern of the electrical characteristic value acquired by the acquirer 41 and the temperature variation pattern of the sensitive unit 2 detected by the temperature sensor to identify a type of an odor gas (in Identification step ST4).

When the type of the odor gas is identified by the identifier 43 of the second sensor module 1L as the slave, the outputter 44 of the slave allows the communications unit 52 of the slave to transmit the identification result of the identifier 43 of the slave to the first sensor module 1R as the master. When the communications unit 52 of the first sensor module 1R as the master receives the identification result of the identifier 43 of the second sensor module 1L as the slave from the second sensor module 1L, the identifier 43 of the first sensor module 1R identifies the type of the odor gas and specifies the direction or the area in which the odor gas is present based on: the identification result decided based on the output signal acquired by the acquirer 41 of the first sensor module 1R; and the identification result of the identifier 43 of the second sensor module 1L. That is to say, the identifier 43 specifies at least one of the direction in which the odor gas is present or the area where the odor gas is present based on: the result obtained by inputting the output signal of the sensitive unit 2 (the first sensitive unit) included in the first sensor module 1R to the learned model MD1; and the result obtained by inputting the output signal of the sensitive unit 2 (the second sensitive unit) included in the second sensor module 1L to the learned model MD1. If no odor gas has been detected even by both the first sensor module 1R and the second sensor module 1L, the identifier 43 of the first sensor module 1R decides that a detectable odor gas is not present around the user 100 and does not notify the user 100.

If any odor gas has been detected by least one of the first sensor module 1R or the second sensor module 1L, the processor 40 of the first sensor module 1R outputs the notification information to the user 100. If the same types of odor molecules have been detected by both the first sensor module 1R and the second sensor module 1L, the processor 40 of the first sensor module 1R decides that the odor gas is present in the detection areas AR1 and AR2 of the first sensor module 1R and the second sensor module 1L. The processor 40 of the first sensor module 1R allows the outputter 44 to output, to the notification module 5, the notification information for notifying the user of the type of the odor gas thus detected and the direction or the area in which the odor gas is present. In the notification module 5 of the first sensor module 1R, the loudspeaker 51 outputs the notification information by voice and the communications unit 52 transmits the notification information to the second sensor module 1L as the slave. In the second sensor module 1L as the slave, when the communications unit 52 receives the notification information from the first sensor module 1R, the processor 40 allows the outputter 44 to output the notification information to the loudspeaker 51 of the notification module 5, thereby the notification information being output by voice from the loudspeaker 51. Accordingly, the notification information is audio output from the loudspeakers 51 of both the first sensor module 1R and the second sensor module 1L.

When only either the first sensor module 1R or the second sensor module 1L has detected the odor gas, the processor 40 of the first sensor module 1R allows the outputter 44 to output, to the notification module 5, the notification information for notifying the user of the type of the odor gas thus detected and the direction or the area (the detection area AR1 or AR2) in which the odor gas is present. In the notification module 5 of the first sensor module 1R, the loudspeaker 51 outputs the notification information by voice and the communications unit 52 transmits the notification information to the second sensor module 1L as the slave. In the second sensor module 1L as the slave, when the communications unit 52 receives the notification information from the first sensor module 1R, the processor 40 allows the outputter 44 to output the notification information to the loudspeaker 51 of the notification module 5, thereby the notification information being output by voice from the loudspeaker 51. Accordingly, even when only either the first sensor module 1R or the second sensor module 1L has detected the odor gas, the notification information is audio output from the loudspeakers 51 of both the first sensor module 1R and the second sensor module 1L.

In this embodiment, each of the two or more sensor modules 1 includes an identifier 43 but should not be construed as limiting. Alternatively, only one sensor module 1 serving as the master, out of the two or more sensor modules 1, may include an identifier 43, for example. In this case, the single identifier 43 of the one sensor module 1 may identify a type of an odor gas based on output signals of two or more sensitive units 2.

### (3) Variations

Note that the embodiment described above is only an exemplary one of various embodiments of the present disclosure and should not be construed as limiting. Rather, the exemplary embodiment may be readily modified in various manners depending on a design choice or any other factor without departing from the scope of the present disclosure. Also, the functions of the odor detection system 10 may also be implemented as, for example, an odor detection method, a computer program, or a non-transitory storage medium on which the program is stored. An odor detection method according to an aspect includes an acquisition step, an identification step and an output step. The acquisition step includes acquiring, as an output signal of a sensitive unit 2, data about an electrical characteristic value of the sensitive unit 2 from a sensor module 1. The sensor module 1 includes: the sensitive unit 2 having the electrical characteristic value varying in response to that the sensitive unit 2 is exposed to an odor gas; and a housing 60 in which the sensitive unit 2 is housed, the housing 60 being attached to a user 100. The identification step includes inputting, to a learned model MD1, the output signal acquired in the acquisition step to identify a type of the odor gas. The learned model MD1 is generated by machine learning using, as input data, two or more output signals of the sensitive unit 2 obtained in two or more states where the sensitive unit 2 is exposed to two or more types of odor gases, respectively. The output step includes outputting notification information representing an identification result obtained in the identification step. A (computer) program according to another aspect is designed to cause a computer system to perform the acquisition step, the identification step, and the output step.

Hereinafter, variations of the above-described exemplary embodiment will be enumerated one after another. Note that the variations to be described below may be adopted in combination as appropriate. Any constituent element of the following variations, having the same function as a counterpart of the embodiment described above, will be designated by the same reference numeral as that counterpart's, and description thereof will be omitted herein.

The odor detection system 10 or the odor detection method according to the present disclosure includes a computer system as an executing subject, for example. The computer system may include a processor and a memory as principal hardware components thereof. The functions as the executing subject of the odor detection system 10 or the odor detection method according to the present disclosure may be performed by making the processor execute a program stored in the memory of the computer system. The program may be stored in advance in the memory of the computer system. Alternatively, the program may also be downloaded through a telecommunications line or be distributed after having been recorded in some non-transitory storage medium such as a memory card, an optical disc, or a hard disk drive, any of which is readable for the computer system. The processor of the computer system may be made up of a single or a plurality of electronic circuits including a semiconductor integrated circuit (IC) or a large-scale integrated circuit (LSI). As used herein, the “integrated circuit” such as an IC or an LSI is called by a different name depending on the degree of integration thereof. Examples of the integrated circuits include a system LSI, a very-large-scale integrated circuit (VLSI), and an ultra-large-scale integrated circuit (ULSI). Optionally, a field-programmable gate array (FPGA) to be programmed after an LSI has been fabricated or a reconfigurable logic device allowing the connections or circuit sections inside of an LSI to be reconfigured may also be adopted as the processor. Those electronic circuits may be either integrated together on a single chip or distributed on multiple chips, whichever is appropriate. Those multiple chips may be aggregated together in a single device or distributed in multiple devices without limitation. As used herein, the “computer system” includes a microcontroller including one or more processors and one or more memories. Thus, the microcontroller may also be implemented as a single or a plurality of electronic circuits including a semiconductor integrated circuit or a large-scale integrated circuit.

Also, it is not essential that the plurality of functions of the odor detection system 10 are integrated together in a single housing. Alternatively, those constituent elements of the odor detection system 10 may be distributed in multiple different housings. Still alternatively, at least some functions of the odor detection system 10 (e.g., some functions such as of the odor detection system 10; more specifically e.g., a learned model MD1, an identifier 43, etc.) may be implemented as a cloud computing system as well. For example, the identifier 43 may use the learned model MD1 located on the cloud computing to identify a type of an odor gas. That is to say, the identifier 43 of the odor detection system 10 may input, to the learned model on the cloud computing, the output signal representing the variation pattern of the electrical characteristic value (resistance value), obtained in the state where the temperature of the sensitive unit 2 exposed to the odor gas is caused to vary in the temperature variation pattern described above, to acquire the identification result from the learned model on the cloud computing, thereby identifying the state of the odor gas.

### (3.1) First Variation

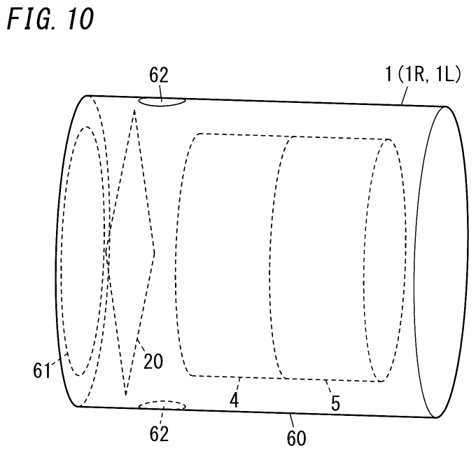

FIG. 10 illustrates a schematic perspective view of an appearance of a sensor module 1 (a first sensor module 1R and a second sensor module 1L) included in an odor detection system 10 according to the first variation.

The odor detection system 10 according to the first variation differs from that of the embodiment described above in that the sensor module 1 does not include the air blower 7. In the odor detection system 10 according to the first variation, air flows into the housing 60 through the intake port 61 by natural air flow, and is then exhausted through the outlet port 62, so that the sensitive unit 2 can be exposed to air outside the housing 60.

In the odor detection system 10 according to the first variation, an air flow may be generated such that air flows into the housing 60 through the outlet port 62 and is exhausted through the intake port 61. Even in this case, the sensitive unit 2 can be exposed to air outside the housing 60. That is to say, an air flow may be generated in the interior of the housing 60 such that air flows into the housing 60 through a first hole, out of a plurality of holes including the intake port 61 and the outlet port 62 and is exhausted through a second hole, out of the plurality of holes, so that the sensitive unit 2 can be exposed to air outside the housing 60.

### (3.2) Second Variation

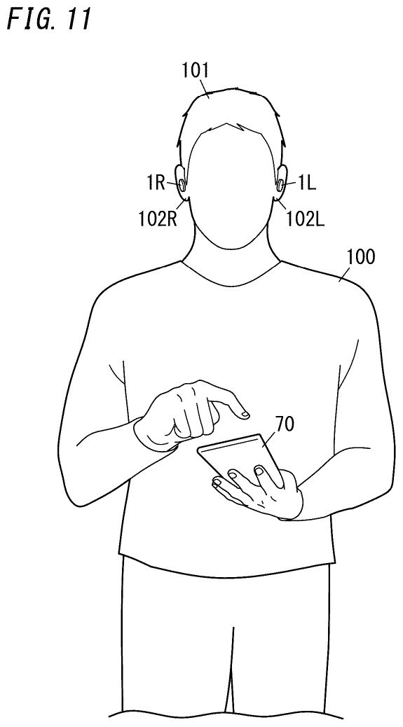

FIG. 11 illustrates a use state of an odor detection system 10 according to the second variation.

The odor detection system 10 according to the second variation differs from that of the embodiment described above in that the outputter 44 allows the communications unit 52 of the notification module 5 to wirelessly transmit the notification information to a mobile terminal 70 having a communication function. In other words, in the odor detection system 10 according to the second variation, the outputter 44 is configured to allow the communications unit 52, configured to communicate with the mobile terminal 70 having the communications function, to transmit the notification information to the mobile terminal 70. The mobile terminal 70 is implemented as a mobile terminal device having a communications function, such as a smartphone or a tablet computer.

For example, the outputter 44 may transmit, to the mobile terminal 70, the notification information for outputting the identification result for the odor gas by voice or outputting it by texts or diagrams. Thus, the mobile terminal 70 may output the detection result for the odor gas, obtained by the odor detection system 10, by voice from a loudspeaker of the mobile terminal 70, or display the detection result by texts or diagrams on a display device of the mobile terminal 70. For example, the mobile terminal 70 may graphically display a direction in which the odor gas is present or an area where the odor gas is present, thereby the notification information being presented to the user 100 in an easy-to-understand manner.

### (3.3) Third Variation

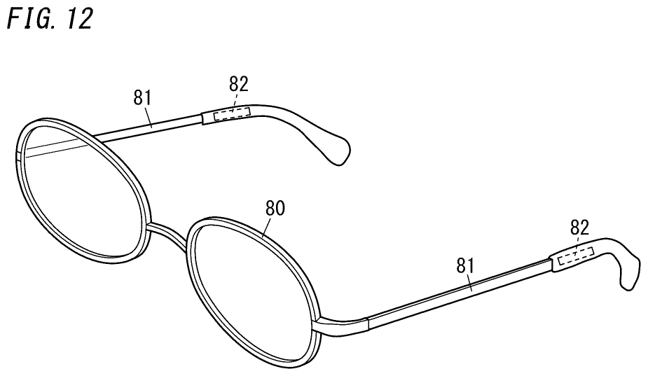

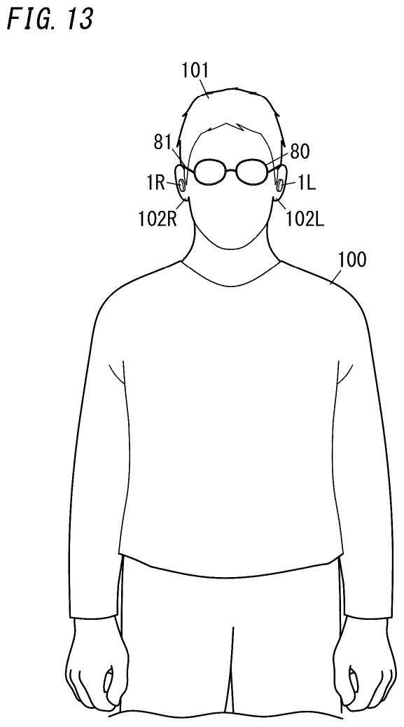

An odor detection system 10 according to the third variation differs from that of the embodiment described above in that the notification information is audio output using an eyeglasses-type notification module 80. FIG. 12 illustrates a perspective view of an appearance of the eyeglasses-type notification module 80. FIG. 13 illustrates a use state of the odor detection system 10 according to the third variation.

The eyeglasses-type notification module 80 includes temples 81 and bone conduction speakers 82 respectively incorporated into the temples 81 to output voice. The notification module 80 further includes a communications part configured to communicate with the communications units 52 of the sensor module 1 and respectively mounted in the frames of the notification module 80. When the communications part of the notification module 80 receive the notification information from the communications units 52 of the sensor module 1, the bone conduction speakers 82 output a voice based on the notification information. Therefore, the user 100 can hear the detection result information about the odor gas by voice.

In the odor detection system 10 according to the third variation, the housing 60 of the sensor module 1 in which the sensitive unit 2 is housed is attached in the ear hole. However, it is not essential that the sensitive unit 2 be disposed in the ear hole. In other words, as long as the sensitive unit 2 is attached to the body of the user 100, it may be mounted at any position.

### (3.4) Fourth Variation

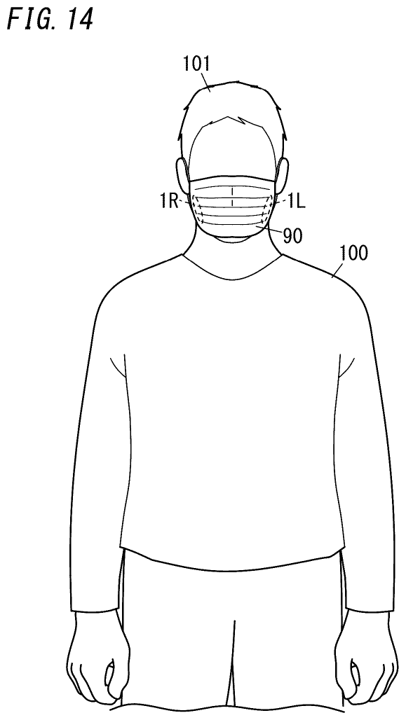

An odor detection system 10 according to the fourth variation differs from that of the embodiment described above in that the first sensor module 1R and the second sensor module 1L are incorporated in a face mask 90 to be mounted by the user 100 (refer to FIG. 14).

The first sensor module 1R and the second sensor module 1L incorporated in the face mask 90 differ from those of the embodiment described above in that no air blower 7 is provided.

The sensitive unit 2 is disposed on, for example, a surface of the face mask 90 to be exposed to air. The electrical characteristic value may accordingly vary depending on a type and a concentration of an odor gas present around the user 100. Thus, the identifier 43 may input the output signal of the sensitive unit 2 into the learned model MD1 to identify the type of the odor gas present around the user 100.

### (3.5) Other Variations

In the embodiment described above, the housing 60 of the sensor module 1 is attached to the body of the user 100 with the housing 60 partially inserted into a corresponding ear hole of the user 100. However, this is only an example and should not be construed as limiting. Alternatively, the housing 60 of the sensor module 1 may be implemented as a hook-type housing to be hooked to the ear of the user, or a headphone-type housing to be mounted on the head 101 of the user.

The housing 60 of the sensor module 1 may include intake ports respectively disposed in a plurality of directions. The direction or area of the odor gas (to which the sensitive unit 2 is exposed) can be switched by switching any intake port to be used to draw air outside the housing 60 among the intake ports. Therefore, the sensor module 1 can more accurately detect the direction or area in which the odor gas is present.

The sensor module 1 is directly attached to the body of the user 100 in the embodiment described above. However, this is only an example and should not be construed as limiting. Alternatively, the sensor module 1 may be attached to clothing, a hat or the like, worn by the user 100. That is to say, the user 100 wears the clothing, hat, or the like to which the sensor module 1 is attached, so that the sensor module 1 may be attached to the user 100.

In the odor detection system 10 of the embodiment described above, the sensor module 1 includes the heater 3 for heating the sensitive unit 2. However, this is only an example and should not be construed as limiting. Alternatively, the sensor module 1 may include a Peltier element capable of both heating and cooling the sensitive unit 2.

In the odor detection system 10 of the embodiment described above, the sensitive unit 2 includes the sixteen sensitive elements Ax. However, this is only an example and should not be construed as limiting. The number of sensitive elements Ax may be changed as needed. The sensitive unit 2 includes both the negative characteristic sensitive elements and the positive characteristic sensitive elements. However, this is only an example and should not be construed as limiting. Alternatively, the sensitive unit 2 may include only a negative characteristic sensitive element(s) or may include only a positive characteristic sensitive element(s). In the odor detection system 10 of the embodiment described above, the sixteen sensitive elements Ax are arranged in four rows and four columns. However, the arrangement of the plurality of sensitive elements Ax is not limited to such an arrangement in the embodiment described above. Alternatively, the plurality of sensitivity elements may be arranged in a line-like manner or may be arranged in a spaced-apart arrangement on one or more concentric circles.

### (Recapitulation)

As described above, an odor detection system (10) according to a first aspect includes a sensor module (1, 1R, 1L), an acquirer (41), an identifier (43) and an outputter (44). The sensor module (1, 1R, 1L) includes: a sensitive unit (2) having an electrical characteristic value varying in response to that the sensitive unit (2) is exposed to an odor gas; and a housing (60) in which the sensitive unit (2) is housed, the housing (60) being attached to a user (100). The acquirer (41) is configured to acquire, as an output signal of the sensitive unit (2), data about the electrical characteristic value of the sensitive unit (2) from the sensor module (1, 1R, 1L). The identifier (43) is configured to input, to a learned model (MD1), the output signal acquired by the acquirer (41) to identify a type of the odor gas. The learned model (MD1) is generated by machine learning using, as input data, two or more output signals of the sensitive unit (2) obtained in two or more states where the sensitive unit (2) is exposed to two or more types of odor gases, respectively. The outputter (44) is configured to output notification information representing an identification result obtained by the identifier (43).

According to this aspect, the odor detection system (10) can provide assistance in a function about the sense of smell with respect to various types of odor gases.

In an odor detection system (10) according to a second aspect, which may be implemented in conjunction with the first aspect, the housing (60) includes an intake port (61) and an outlet port (62). The housing (60) is provided with an air blower (7). The air blower (7) is disposed to introduce an air outside of the housing (60) into the housing (60) through the intake port (61), and exhaust the air to an outside of the housing (60) through the outlet port (62).

According to this aspect, the odor detection system (10) can reduce in the time required for detecting odor present around the user (100).

In an odor detection system (10) according to a third aspect, which may be implemented in conjunction with the second aspect, the sensor module (1) includes a first sensor module (1R) and a second sensor module (1L). The first sensor module (1R) and the second sensor module (1L) are respectively attached to parts, which are different from each other, of a body of the user (100).

According to this aspect, the odor detection system (10) including the first sensor module (1R) and the second sensor module (1L) can expand an area where the odor gas can be detected.

In an odor detection system (10) according to a fourth aspect, which may be implemented in conjunction with the third aspect, the identifier (43) is further configured to specify at least one of a direction in which the odor gas is present or an area where the odor gas is present based on: the identification result obtained by inputting, to the learned model (MD1), the output signal of the sensitive unit (2) included in the first sensor module (1R); and the identification result obtained by inputting, to the learned model (MD1), the output signal of the sensitive unit (2) included in the second sensor module (1L).

According to this aspect, the user (100) can know not only the type of the odor gas but also at least one of the direction in which the odor gas is present or the area where the odor gas is present.

In an odor detection system (10) according to a fifth aspect, which may be implemented in conjunction with the third to fourth aspect, the first sensor module (1R) and the second sensor module (1L) are respectively attached to right and left ears of the user (100).

According to this aspect, the first sensor module (1R) and the second sensor module (1L) can detect the odor gas present on the right and left sides of the body of the user (100).

In an odor detection system (10) according to a sixth aspect, which may be implemented in conjunction with any one of the first to fifth aspects, the outputter (44) is configured to allow first and second voice output units (51), respectively disposed to the right and left ears (102R, 102L) of the user (100), to output the notification information.

According to this aspect, the user (100) can hear the respective pieces of the notification information output from the first and second voice output units (51) with the right and left ears (102R, 102L).

In an odor detection system (10) according to a seventh aspect, which may be implemented in conjunction with any one of the first to sixth aspects, the outputter (44) is configured to allow a communications unit (52), configured to communicate with a mobile terminal (70) having a communications function, to transmit the notification information to the mobile terminal (70).

According to this aspect, the user (100) can confirm, using the mobile terminal (70), the contents of notification information.

In an odor detection system (10) according to an eighth aspect, which may be implemented in conjunction with any one of the first to seventh aspects, the acquirer (41), the identifier (43) and the outputter (44) are further housed in the housing (60) of the sensor module (1, 1R, 1L).

According to this aspect, the odor detection system (10) can be provided, where the sensor module (1, 1R, 1L), the acquirer (41), the identifier (43) and the outputter (44) are housed in the single housing (60).

An odor detection method according to a ninth aspect includes an acquisition step, an identification step and an output step. The acquisition step includes acquiring, as an output signal of a sensitive unit (2), data about an electrical characteristic value of the sensitive unit (2) from a sensor module (1, 1R, 1L). The sensor module (1, 1R, 1L) includes: the sensitive unit (2) having the electrical characteristic value varying in response to that the sensitive unit (2) is exposed to an odor gas; and a housing (60) in which the sensitive unit (2) is housed, the housing (60) being attached to a user (100). The identification step includes inputting, to a learned model (MD1), the output signal acquired in the acquisition step to identify a type of the odor gas. The learned model (MD1) is generated by machine learning using, as input data, two or more output signals of the sensitive unit (2) obtained in two or more states where the sensitive unit (2) is exposed to two or more types of odor gases, respectively. The output step includes outputting notification information representing an identification result obtained in the identification step.

According to this aspect, the odor detection method can provide assistance in a function about the sense of smell with respect to various types of odor gases.

Various configurations (including variations) of the odor detection system (10) according to the embodiment described above are not limited to the aspects described above, but may also be implemented as, for example, an odor detection method, a (computer) program, or a non-transitory storage medium that stores the program thereon.

Note that the constituent elements according to the second to eighth aspects are not essential constituent elements for the odor detection system (10) but may be omitted as appropriate.

### REFERENCE SIGNS LIST

1 Sensor module
1R First sensor module
1L Second sensor module
2 Sensitive unit (first sensitive unit, second sensitive unit)
7 Air blower
10 Detection system
41 Acquirer
43 Identifier
44 Outputter
51 Loudspeaker (first voice output unit, second voice output unit)
52 Communications unit
60 Housing (first housing, second housing)
61 Intake port
62 Outlet port
70 Mobile terminal
100 User
102R Right ear
102L Left ear
MD1 Learned model

1. An odor detection system comprising:
a sensor module including
a sensitive unit having an electrical characteristic value varying in response to that the sensitive unit is exposed to an odor gas, and
a housing in which the sensitive unit is housed, the housing being attached to a user;
an acquirer configured to acquire, as an output signal of the sensitive unit, data about the electrical characteristic value of the sensitive unit from the sensor module;
an identifier configured to input, to a learned model, the output signal acquired by the acquirer to identify a type of the odor gas, the learned model being generated by machine learning using, as input data, two or more output signals of the sensitive unit obtained in two or more states where the sensitive unit is exposed to two or more types of odor gases, respectively; and
an outputter configured to output notification information representing an identification result obtained by the identifier.

2. The odor detection system of claim 1, wherein
the housing includes an intake port and an outlet port, and
the housing is provided with an air blower, the air blower being disposed to introduce an air outside of the housing into the housing through the intake port, and exhaust the air to an outside of the housing through the outlet port.

3. The odor detection system of claim 2, wherein
the sensor module includes a first sensor module and a second sensor module, and
the first sensor module and the second sensor module are respectively attached to parts, which are different from each other, of a body of the user.

4. The odor detection system of claim 3, wherein
the identifier is further configured to specify at least one of a direction in which the odor gas is present or an area where the odor gas is present based on:
the identification result obtained by inputting, to the learned model, the output signal of the sensitive unit included in the first sensor module; and
the identification result obtained by inputting, to the learned model, the output signal of the sensitive unit included in the second sensor module.

5. The odor detection system of claim 3, wherein
the first sensor module and the second sensor module are respectively attached to right and left ears of the user.

6. The odor detection system of claim 1, wherein
the outputter is configured to allow a first voice output unit and a second voice output unit, respectively disposed to the right and left ears of the user, to output the notification information.

7. The odor detection system of claim 1, wherein
the outputter is configured to allow a communications unit, configured to communicate with a mobile terminal having a communications function, to transmit the notification information to the mobile terminal.

8. The odor detection system of claim 1, wherein
the acquirer, the identifier and the outputter are further housed in the housing of the sensor module.

9. An odor detection method comprising:
an acquisition step including acquiring, as an output signal of a sensitive unit, data about an electrical characteristic value of the sensitive unit from a sensor module,
the sensor module including
the sensitive unit having the electrical characteristic value varying in response to that the sensitive unit is exposed to an odor gas, and
a housing in which the sensitive unit is housed, the housing being attached to a user,
an identification step including inputting, to a learned model, the output signal acquired in the acquisition step to identify a type of the odor gas, the learned model being generated by machine learning using, as input data, two or more output signals of the sensitive unit obtained in two or more states where the sensitive unit is exposed to two or more types of odor gases, respectively; and
an output step including outputting notification information representing an identification result obtained in the identification step.

10. The odor detection system of claim 4, wherein
the first sensor module and the second sensor module are respectively attached to right and left ears of the user.

11. The odor detection system of claim 2, wherein
the outputter is configured to allow a first voice output unit and a second voice output unit, respectively disposed to the right and left ears of the user, to output the notification information.

12. The odor detection system of claim 3, wherein
the outputter is configured to allow a first voice output unit and a second voice output unit, respectively disposed to the right and left ears of the user, to output the notification information.

13. The odor detection system of claim 4, wherein
the outputter is configured to allow a first voice output unit and a second voice output unit, respectively disposed to the right and left ears of the user, to output the notification information.

14. The odor detection system of claim 5, wherein
the outputter is configured to allow a first voice output unit and a second voice output unit, respectively disposed to the right and left ears of the user, to output the notification information.

15. The odor detection system of claim 2, wherein
the outputter is configured to allow a communications unit, configured to communicate with a mobile terminal having a communications function, to transmit the notification information to the mobile terminal.

16. The odor detection system of claim 3, wherein
the outputter is configured to allow a communications unit, configured to communicate with a mobile terminal having a communications function, to transmit the notification information to the mobile terminal.

17. The odor detection system of claim 4, wherein
the outputter is configured to allow a communications unit, configured to communicate with a mobile terminal having a communications function, to transmit the notification information to the mobile terminal.

18. The odor detection system of claim 5, wherein
the outputter is configured to allow a communications unit, configured to communicate with a mobile terminal having a communications function, to transmit the notification information to the mobile terminal.

19. The odor detection system of claim 6, wherein
the outputter is configured to allow a communications unit, configured to communicate with a mobile terminal having a communications function, to transmit the notification information to the mobile terminal.
